(19) United States
(12) Patent Application Publication
(10) Pub. No.: US 2006/0115880 A1
(43) Pub. Date: Jun. 1, 2006

(54) Enzymatic production of acyl flavonoid derivatives

(75) Inventors: Mohamed Ghoul, Nancy (FR); Jean-Marc Engasser, Ludres (FR); Philippe Moussou, Nancy (FR); Gilles Pauly, Nancy (FR); Melika Ardhaoui, vandoeuvre-Les-Nancy (FR); Aude Falcimaigne, Nancy (FR)

Correspondence Address: COGNIS CORPORATION;PATENT DEPARTMENT, 300 BROOKSIDE AVENUE, AMBLER, PA, 19002, US

(73) Assignee: Cognis France S.A., Saint-Martory (FR)

(21) Appl. No.: 10/537,627

(22) PCT Filed: Nov. 22, 2003
(86) PCT No.: PCT/EP03/13143

§ 371 Date: Jun. 3, 2005

(30) Foreign Application Priority Data
Dec. 3, 2002 (EP) 02292969.9

(51) Int. Cl. C12P 17/06

(52) U.S. Cl. 435/125

(57) ABSTRACT

Disclosed is a method for enzymatic synthesis of esters of flavonoid and flavonoid derivatives, according to which a reaction medium containing an organic solvent, a glycosylated flavonoid or aglycon flavonoid, an acyl group donor, and an enzymatic catalyst is produced, b) additional quantities of flavonoid and/or acyl donor are optionally added during the reaction, and c) the obtained esters are separated from the enzymatic particles and the solvent. The inventive method is characterized by maintaining the concentration of water and/or alcohol which are formed during the reaction to below 150 mM. Advantageously, the molar ratio of flavonoid to acyl donor in the reaction medium is maintained in the range from 0.01 to 20.00 during the reaction.

### FIELD OF THE INVENTION

This invention relates generally to phyto- and biochemistry and, more particularly, to a process for the enzymatic production of flavonoid derivatives for use in foods and in cosmetic and pharmaceutical preparations.

### PRIOR ART

The biological effects of the flavonoids have been well-known for many years. By trapping various oxidizing species, they prevent oxidative damage to biomolecules, such as DNA, lipids and proteins. In antioxidant assays, some flavonoids are more effective than vitamins C and E. Apart from this main property, several other biological effects have been demonstrated, including inhibition of the effect of enzymes and the proliferation of animal cells, viruses and bacteria. They also have an effect on the vascular system and a considerable antioxidative capacity.

By virtue of their skin-protecting and skin-cleansing properties and their effects against ageing, against skin discoloration and on the appearance of the skin, flavonoids have also been used as constituents of cosmetic or dermopharmaceutical compositions. They also act on the mechanical properties of the hair.

The antioxidation properties of the flavonoids depend upon their molecular structure. Investigation of the structure/effect relationship has shown that the antioxidative effect is based on an ortho-hydroxylation at the ring B of the molecule, the number of free hydroxyl groups, the presence of a double bond between carbons 2 and 3 in the ring C and the presence of a hydroxyl group at carbon 3 (FIG. 1).

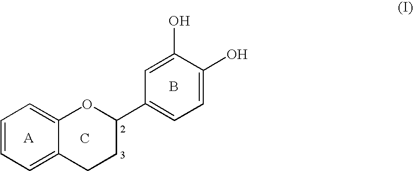

The use of these molecules is basically limited on the one hand by very poor solubility in both aqueous and organic media and, on the other hand, by poor stability. Flavonoids are degraded by light, oxygen or oxidizing agents and increases in temperature. These limitations prevent their effective use in foods and cosmetic and pharmaceutical compositions.

There are various well-known strategies for eliminating this stability problem, including encapsulation and formulation with antioxidants. Unfortunately, none of these solutions is entirely satisfactory and there is still a need for new glycosylated flavonoids and aglyconflavonoids with improved stability.

Enzymatic and chemical modifications have been proposed with a view to improving the properties of these molecules. Thus, JP 55157580 and JP 58131911 mention the acylation of quercetin with fatty acid chlorides in dioxan in the presence of pyridine. In these patents, however, the acylation is carried out in the presence of toxic solvents. The substrate conversion yields are low. The acylation of flavone, flavonol and flavanone in the same way is described in the Coletica patent FR 2778663 (U.S. Pat. No. 6,235,294). This reaction was carried out chemically in the presence of a fatty acid chloride or anhydride. These reactions involve the use of activated fatty acids and toxic solvents (pyridine, chloroform and toluene) and also high temperatures (100° C.). The substrate conversion yields are low (ca. 10 to 60%). In addition, these reactions are not selective which leads to polyacylated products. WO 09966062 mentions the chemical acylation of flavonoids (quercetin, galangin, (+)-catechol) by fatty acids (laurylic acid, butyric acid, acetic acid . . . ) and a following enzymatic hydrolysis step by a lipase of Mucor miehei. This invention has the same disadvantages as FR 2778663 (U.S. Pat. No. 6,235,294). After the initial acylation reaction, the products formed are polyacylated. If monoesters are required, an enzymatic hydrolysis has to be carried out in a second step after removal of the solvents used in the first reaction in order to avoid deactivation of the enzyme. Flavonoid modifications are also described in EP 0618203 in which the acylation of (±)-catechol by ethyl acetate and ethyl propionate and the acylation of epigallolcatechol by phenyl propionate and butyrate are mentioned. This reaction requires an expensive enzyme (the carboxylase of Streptomyces rochei) and the conversion yield of the two substrates is very low (less than 1% of the acyl donor). Finally, WO 0179245 (Henkel/Cognis) describes the enzymatic acylation of flavonoids (naringin, rutin, asparatin, orientin, quercetin, kaempferol, cis-orientin, isoquercitrin) by various acids (p-chlorophenylacetic acid, stearic acid, 12-hydroxystearic acid, palmitic acid, lauric acid, capric acid, 4-hydroxyphenylacetic acid, 5-phenylvaleric acid, coumaric acid, oleic acid, linoleic acid). This patent describes a process in which a high concentration of Candida antarctica (40 g/l) and—based on the flavonoids—an excess of acyl donor are used. The conversion yield of the substrates is low (10 to 20%).

All the works described above are characterized by low yields. In addition, the chemical modifications involve the use of toxic solvents, the reactions are non-specific which leads to product mixtures and the described enzymatic reactions are limited to glycosylated flavonoids. The low conversion yields mentioned in the prior art are attributable to the fact that the processes used are not adapted to reactions with poorly soluble substrates and are seriously impaired by the water formed during the reaction.

### DESCRIPTION OF THE INVENTION

The present invention relates to a process for the enzymatic synthesis of flavonoid esters and derivatives in which
a) a reaction medium containing an organic solvent, a glycosylated flavonoid or aglycon flavonoid, an acyl group donor and an enzymatic catalyst is prepared,
b) further quantities of flavonoid and/or acyl donor are optionally added during the reaction and
c) the esters thus obtained are purified by removing enzymatic particles and the solvent,
   characterized in that the concentration of water and/or alcohol formed during the reaction is controlled so that it remains below 150 mM.

The present invention—a process for the selective acylation of glycosylated flavonoids and aglycon flavonoids—leads to an improvement in the flavonoid derivatives in terms of their stability and solubility in various preparations, their antioxidative properties remaining intact or being improved. Another particular advantage obtained by these modified flavonoids is that bifunctional molecules with higher biological activity are formed.

By comparison with the methods known from the prior art, a distinct improvement in regard to the final concentration of flavonoid esters, the conversion yields (both for the flavonoids and for the acyl donor originally present) and, in particular, productivity can be obtained by the process according to the invention while the complicated and elaborate purifying operations after the synthesis are reduced.

The process according to the invention is based on an enzyme technology using mild temperature and pressure conditions, but no dangerous solvents, the flavonoid esters being formed by direct esterification or transesterification in accordance with the following reaction schemes:
Flavonoid+RCOOH→Flavonoid-OCOR+H₂O
Flavonoid+RCOOR′→Flavonoid-OCOR+R′OH
in which R′ is a C₁₋₄ alkyl group, preferably a C₁₋₂ alkyl group.

This process is characterized in that the reaction medium is first freed from water, so that water present before the beginning of the reaction is removed, and the water or alcohol formed during the reaction is removed on-line. Water and/or alcohol are kept at concentrations suitable for the solvents and substrates used, preferably at concentrations below 150 mM and more particularly at concentrations below 100 mM.

The process is suitable for a number of aglycon flavonoids and glycosylated flavonoids and the conversion yields obtained with this mild enzymatic process are above those hitherto obtained, namely in the range from 50 to 99%.

The enzymatic synthesis is carried out under milder conditions than the chemical syntheses and avoids the use of toxic solvents, such as pyridine, benzene and THF, high temperatures and the formation of secondary products, such as salts or flavonoid degradation products which would necessitate additional purification steps.

By comparison with the known methods mentioned above, a distinct improvement in regard to the final concentration of flavonoid esters, the conversion yields (both for the flavonoids and for the acyl donor originally present) and, in particular, productivity can be obtained with this process while the complicated and elaborate purifying steps after the synthesis are reduced.

One difference between the process according to the invention and the known methods lies in the manner in which the enzymatic process is carried out, which provides for considerably higher yields, and in the large number of different flavonoids which can be used (both glycosylated forms and aglycon forms).

The principal object of the invention is to reduce all the above-mentioned disadvantages of existing acylation methods and to provide a process for the enzymatic synthesis of flavonoid esters which, by comparison with the known methods mentioned above, would allow a distinct improvement in regard to the final concentration of flavonoid esters, the conversion yields (both for the flavonoids and for the acyl donor originally present) and, in particular, productivity to be obtained while reducing the complicated and elaborate purifying steps after the synthesis.

To this end, the present invention relates to a process for the enzymatic synthesis of flavonoid esters which is characterized in that, to prepare a reaction medium, predetermined quantities of a flavonoid (glycosylated forms and aglycon forms) or flavonoid derivative, an acyl group donor, an organic solvent—which may be the acyl donor—and an enzymatic catalyst are introduced into a correspondingly designed reactor under conditions where firstly the reaction medium can be dried to a water concentration below 150 mM and preferably to a water concentration below 100 mM and the concentration of water and/or alcohol formed during the reaction can be kept below a predetermined value of 150 mM, preferably 100 mM. The concentration is kept to this predetermined value by on-line removal of the water and/or alcohol formed by adsorption onto molecular sieves, by distillation or by pervaporation. This reaction can be carried out as a batch process or even as a fed batch process with one or more substrates. In the fed batch process, the molar ratio of flavonoid to acyl donor can be kept constant by a suitable substrate addition profile during the reaction. It is thus possible to control how the composition of the reaction medium develops as a function of time and, hence, to steer the enzymatic reaction towards maximum production of mono- or multiacylated compounds and, at the same time, to limit troublesome reactions. Finally, the flavonoid esters thus obtained are purified by at least removing enzymatic particles (for example by decantation, filtration or centrifuging) and the solvent (for example by evaporation, distillation or membrane filtration).

According to the invention, the reaction is carried out by first limiting the inhibition or deactivation of the enzyme reaction which is observed in the presence of high concentrations of flavonoids, acyl donors or accumulations of water. The substrates may be gradually added under control during the reaction so that concentrations that would inhibit the enzyme reaction are never reached.

The reaction may be carried out with a flavonoid:acyl donor molar ratio of 0.01 to 20:1 and preferably 0.02 to 10:1. By keeping the values for the molar ratio in the reaction medium in the ranges mentioned above, it is possible to obtain either higher reaction rates or maximum percentages of monoacylated flavonoids. By controlling the type and quantity of reagents added as a function of time, the molar ratio can be kept constant or varied under control during the reaction so that it passes through a certain variation profile as a function of time, but still remains in the above-mentioned range throughout the reaction. The synthesis reaction can be optimized by periodic or continuous removal of at least one constituent of the reaction medium. The constituent(s) removed may be returned to the reactor, possibly after fractionation.

In one embodiment of the invention, the entire reaction medium may be periodically or continuously removed and one or more constituents of the medium removed may be re-injected into the reactor after fractionation.

The reaction vessel or reactor used to carry out the process according to the invention is preferably equipped with means for controlling the temperature, the water and/or alcohol content and the pressure, with means for adding reagents and with means for removing products.

During the synthesis reaction, the temperature is advantageously kept at 20 to 100° C. and the partial pressure over the reaction medium is advantageously adjusted to a value of 10 mbar (10³ Pa) to 1,000 mbar (10⁵ Pa) and, starting from a water content adjusted to concentration below 150 mM and preferably below 100 mM, the quantity of water and/or alcohol is kept below 150 mM and preferably below 100 mM and the reaction medium is advantageously gently stirred.

In addition, to obtain preparations of highly pure flavonoid esters, additional concluding fractionations may be carried out, for example by removing the remaining flavonoids or fats by extraction with organic solvents or supercritical fluids, by distillation or molecular distillation, by precipitation or by crystallization.

The aglycon flavonoid or glycosylated flavonoid or flavonoid derivative used for the purposes of the invention may be any compound selected from the group consisting of chalcone, flavone, flavanol, anthocyan and flavanone, flavanol, coumarin, isoflavones and xanthones.

The acyl donor compound is selected from known fatty acids or methyl, ethyl, propyl or butyl esters thereof. This fatty acid is preferably selected from the group consisting of a linear or branched, saturated, unsaturated or cyclic aliphatic acid containing up to 22 carbon atoms and optionally substituted by one or more substituents selected from the group consisting of hydroxyl, amino, mercapto, halogen and alkyl-S,S-alkyl, for example palmitic acid, 16-hydroxyhexadecanoic acid, 12-hydroxystearic acid, 11-mercaptoundecanoic acid, thiooctanoic acid or quinic acid, linear or branched, saturated or unsaturated aliphatic diacids containing up to 22 carbon atoms, for example hexadecane diacid or azelaic acid, an arylaliphatic acid and a dimeric acid derived therefrom, a cinnamic acid optionally substituted by one or more substituents selected from the group consisting of hydroxyl, nitro, alkyl, alkoxy and halogen atoms, for example coffeic acid (3,4-dihydroxycinnamic acid), ferulic acid (4-hydroxy-3-methoxycinnamic acid) or coumaric acid (4-hydroxycinnamic acid), a benzoic acid optionally substituted by one or more substituents selected from the group consisting of hydroxyl, nitro, alkyl, alkoxy and halogen atoms, for example gallic acid (3,4,5-trihydroxybenzoic acid), vanillic acid (4-hydroxy-3-methoxybenzoic acid) or protocatechu acid (3,4-dihydroxybenzoic acid). The fatty acid esters are preferably selected from the methyl or ethyl esters of the above compounds.

The reaction may be carried out with the acyl donor as solvent or in a suitable solvent which may an organic compound or a mixture of organic compounds in which the selected flavonoids or flavonoid derivatives and acyl donors are completely or partly solubilized. Thus, the solvent(s) is/are selected in particular from the following substances: propan-2-ol, butan-2-ol, isobutanol, acetone, propanone, butanone, pentan-2-one, ethane-1,2-diol, butane-2,3-diol, dioxan, acetonitrile, 2-methylbutan-2-ol, tert.butanol, 2-methylpropanol and 4-hydroxy-2-methylpentanone, aliphatic hydro-carbons, such as heptane, hexane, or a mixture of two or more of these solvents.

The enzymatic catalyst used must of course effect and promote the transfer of an acyl group from an acyl donor to a flavonoid or flavonoid derivative and is advantageously a protease or lipase, for example from Candida antarctica, Rhizomucor miehei, Candida cylindracea, Rhizopus arrhizus, preferably immobilized on a carrier.

By virtue of the various features mentioned above, there are various possible embodiments of the invention, particularly in regard to the nature of the reagents used and the preferred objectives to be achieved.

Accordingly, a first possible embodiment is a synthesis process in a batch reactor (both substrates are introduced into the reactor with solvent and enzyme). In this case, the reactor initially accommodates the solvent, the total quantity of flavonoids (generally from 1 g/l to 200 g/l) needed to obtain the final quantity of modified flavonoids required and the quantity of free acid as acyl donor which corresponds to the originally necessary molar ratio (of dissolved flavonoid/acyl donor) of generally 0.01 to 20:1. In order to obtain a fixed value for the quantity of water in the reactor of under 100 mM, the medium is heated in vacuo (10-500 mbar, preferably 50-250 mbar) to a temperature of 20 to 100° C. and preferably to a temperature of 40 to 80° C. and the vapour mixture produced is dried in a column filled with molecular sieves and then condensed and returned to the reactor. If necessary, the condensate is returned via a second column filled with molecular sieves. The enzyme is then added in soluble or immobilized form (from 1 g/l to 100 g/l and preferably from 5 g/l to 20 g/l). Water formed during the reaction is removed via the column filled with molecular sieves by adjusting the vacuum and the temperature in the reactor accordingly.

Accordingly, a second possible embodiment of the invention is a synthesis process in which the acyl donor and the solvent are added during the reaction. In this case, the reactor initially holds the solvent, the total quantity of flavonoids (generally from 1 g/l to 200 g/l) needed to obtain the final quantity of modified flavonoids required and the quantity of free acid as acyl donor which corresponds to the originally necessary molar ratio (of dissolved flavonoid/acyl donor) of generally 0.01 to 20:1. In order to obtain a fixed value for the quantity of water in the reactor of under 100 mM, the medium is heated in vacuo (10-500 mbar, preferably 50-250 mbar) to a temperature of 20 to 100° C. and preferably to a temperature of 40 to 80° C. and the vapour mixture produced is dried in a column filled with molecular sieves and then condensed and returned to the reactor. If necessary, the condensate is returned via a second column filled with molecular sieves. The enzyme is then added in soluble or immobilized form (from 1 g/l to 100 g/l and preferably from 5 g/l to 20 g/l). During the reaction, solvent is added so that part of the solvent is evaporated via a column filled with molecular sieves. The water is removed by exchange in the vapor phase. The vapor is condensed and collected in a collecting vessel. In order to keep the quantity of solvent relatively constant, acyl donor is introduced on-line during the reaction in such a quantity per unit of time that the molar ratio (of dissolved flavonoid/acyl donor) is kept at the required value. Thus, if it is of advantage to keep this molar ratio constant during the reaction, the acyl donor is added at a rate which corresponds to the rate at which it is consumed in the reaction. This consumption rate can be determined by a preliminary kinetic analysis of the enzyme reaction used. The quantity of acyl donor added per unit of time during the reaction generally amounts to 0.01 to 10 grams acyl donor per hour per gram enzyme catalyst in the reactor.

In a third embodiment of the invention, the synthesis process may also be carried out with addition of flavonoids and solvent. In this case, the reactor initially holds the solvent, the total quantity of free acid as acyl donor (generally from 1 g/l to 500 g/l) needed to obtain the final quantity of modified flavonoids required and the quantity of flavonoid which corresponds to the originally necessary molar ratio (of dissolved flavonoid/acyl donor) (generally from 1 g/l to 200 g/l). In order to obtain a fixed value for the quantity of water in the reactor of under 100 mM, the medium is heated in vacuo (10-500 mbar, preferably 50-250 mbar) to a temperature of 20 to 100° C. and preferably to a temperature of 40 to 80° C. and the vapour mixture produced is dried in a column filled with molecular sieves and then condensed and returned to the reactor. If necessary, the condensate is returned via a second column filled with molecular sieves. The enzyme is then added in soluble or immobilized form (from 1 g/l to 100 g/l and preferably from 5 g/l to 20 g/l). During the reaction, solvent is added and a vacuum is applied so that part of the solvent and the water formed are removed by evaporation. The evaporation rate is adjusted by controlling the vacuum and the temperature accordingly. The vapors formed are passed through a column filled with molecular sieves. The water is removed by the contact with the molecular sieves. After removal of the water, the vapor is condensed and collected in a collecting vessel for subsequent return to the reactor. Water-free solvent is optionally introduced during the reaction to make up for evaporation losses and to keep the quantity of solvent relatively constant. In addition, flavonoid is added in such a quantity per unit of time that the molar ratio (of dissolved flavonoid/acyl donor) is kept at the required value. Thus, if it is of advantage to keep this molar ratio constant during the reaction, the flavonoid is added at a rate which corresponds to the rate at which it is consumed in the reaction. This consumption rate can be determined by a preliminary kinetic analysis of the enzyme reaction used. The quantity of flavonoid added per unit of time during the reaction generally amounts to 0.01 to 10 grams flavonoid per hour per gram enzyme catalyst in the reactor.

In a fourth embodiment of the invention, the synthesis process may also be carried out with addition of flavonoid, acyl donor and solvent. In this fourth case, the reactor initially holds the solvent, a variable concentration of flavonoid (preferably higher than the solubility of the flavonoid in the solvent) and the quantity of free acid as acyl donor which corresponds to the originally necessary molar ratio (of dissolved flavonoid/acyl donor). In order to obtain a fixed value for the quantity of water in the reactor of under 100 mM, the medium is heated in vacuo (10-500 mbar, preferably 50-250 mbar) to a temperature of 20 to 100° C. and preferably to a temperature of 40 to 80° C. and the vapour mixture produced is dried in a column filled with molecular sieves and then condensed and returned to the reactor. If necessary, the condensate is returned via a second column filled with molecular sieves. The enzyme is then added in soluble or immobilized form (from 1 g/l to 100 g/l and preferably from 5 g/l to 20 g/l). During the reaction, solvent is added and a vacuum of 10 to 500 mbar and preferably 100 to 250 mbar is applied. To remove the water, the vapors formed are passed through a column filled with molecular sieves. The vapor is condensed and collected in a collecting vessel. Water-free solvent is optionally introduced during the reaction to make up for evaporation losses and to keep the quantity of solvent relatively constant. In addition, flavonoid is added in such a quantity per unit of time that the molar ratio (of dissolved flavonoid/acyl donor) is kept at the required value. If it is of advantage to keep this molar ratio constant during the reaction, flavonoid and acyl donor are added in quantities per unit of time which correspond to the rate at which they are consumed in the reaction. These consumption rates can be determined by a preliminary kinetic analysis of the enzyme reaction used.

In a fifth embodiment of the invention, the continuous synthesis process may alternatively be carried out with addition and removal of flavonoid, acyl donor and/or solvent and, possibly, enzyme catalyst. In this fifth case, the reactor initially holds the solvent, a variable concentration of flavonoid (preferably higher than the solubility of the flavonoid in the solvent) and the quantity of free acid as acyl donor which corresponds to the originally necessary molar ratio (of dissolved flavonoid/acyl donor). In order to obtain a fixed value for the quantity of water in the reactor of under 100 mM, the medium is heated in vacuo (10-500 mbar, preferably 50-250 mbar) to a temperature of 20 to 100° C. and preferably to a temperature of 40 to 80° C. and the vapour mixture produced is dried in a column filled with molecular sieves and then condensed and returned to the reactor. If necessary, the condensate is returned via a second column filled with molecular sieves. The enzyme is then added in soluble or immobilized form. While the reaction proceeds, substances are continuously or periodically removed from the reaction medium. If the enzyme is present in immobilized form, it may be retained in the reactor. After separation, the solvent and possibly the flavonoid and/or the acyl donor may be returned to the reactor. Water-free solvent is added during the reaction to make up for losses by evaporation and removal. In addition, flavonoid and acyl donor may be added in such quantities per unit of time that the molar ratio of these two constituents is kept at the necessary value. The water is removed through molecular sieves, as described above. After the removal of water, the evaporated solvent is condensed and returned to the reactor. If it is of advantage to keep this molar ratio constant during the reaction, flavonoid and acyl donor are added in quantities per unit of time which correspond to the rate at which they are consumed in the reaction and to their respective removal rates.

In a sixth embodiment, the reaction is carried out as described above, except that the free acid as acyl donor is replaced by its methyl, ethyl, propyl or butyl ester, preferably by its methyl or ethyl ester. The alcohol formed is removed in the same way as before.

In a seventh embodiment, the acyl donor is used as solvent.

In an eighth embodiment, water and/or alcohol present in the medium and/or formed therein during the reaction is removed in the vapor or liquid phase by a pervaporation membrane.

### EXAMPLES

### Example 1

The synthesis of rutin monopalmitate was carried out in a 250 ml batch reactor using Candida antarctica lipase (Novozym 435). This is a lipase immobilized on a macroporous acrylic resin. The lipase is supplied with an activity of 7,000 PLU×g⁻¹ (propyl laurate synthesis), a water content of 1-2% by weight and an enzymatic protein content of 1 to 10% by weight.

0.75 g (1.2 mmol) rutin, 0.315 g (1.2 mmol) palmitic acid and 250 ml tert.amylalcohol were used for this synthesis. The medium was heated in vacuo (150 mbar) to 60° C. and the vapor formed was passed through a column heated to 60° C. and filled with 50 g molecular sieves. The water present was thus removed in the gas phase which was far more effective than in the liquid phase. The water-free vapor was condensed and returned to the reactor via a second column which was filled with the same quantity of molecular sieves. In this way, a starting water content of less than 100 mM was obtained after 6 h and the substrates were solubilized. The enzyme (2.5 g) was then added. The reaction was carried out in vacuo (150 mbar) at 60° C. and the water formed was removed in the same way as for the original drying, so that its concentration was kept below 100 mM.

This concentration can be varied by adjusting the vacuum and cooling the condenser accordingly. The pressures investigated varied between 10 and 700 mbar and the temperature of the condenser between −20 and 5° C. It this way, the water concentration in the reactor could be adjusted to between 5 and 400 mM.

After reaction for 48 hours, product analysis by HPLC showed that the conversion yields for the two substrates amounted to ca. 90%.

At the end of the reaction, the enzyme was recovered by filtration. The medium was then concentrated by evaporation of the solvent. To eliminate substrate residues, two extraction systems were used. A mixture of acetonitrile and heptane (3:5, v:v) was used to remove the palmitic acid whereas the rutin was removed with water/heptane (2:3, v:v).

The structure of the product was confirmed by ¹H-NMR analysis:

¹H-NMR: (400 MHz, DMSO-d₆): δ 0.8 (t, 3H), 1 (d, 3H), 1.25 (m, 24H), 1.45 (m, 2H), 2.1 (m, 2H), 3.1-3.6 (broad, C—H sugar), 3.7 (d, 1H), 4.45 (s, 1H), 4.65 (t, 1H), 5.3 (broad, OH sugar), 5.1 (broad, OH sugar), 5.45 (d, 1H), 6.2 (s,1 H), 6.4 (s, 1H), 6.8 (d, 1H), 7.6 (m, 2H), 12.6 (s, 1H, C₅—OH) ppm.

### Example 2

The acylation of hesperidin (0.75 g, 1.2 mmol) with palmitic acid (0.315 g, 1.2 mmol) was carried out as described above.

HPLC analysis showed that 95% of the acyl donor had been consumed after 48 hours. Using the same purification procedure as before, the hesperidin monopalmitate was obtained by liquid/liquid extraction. The structure of the hesperidin ester was confirmed by ¹H-NMR analysis:

¹H-NMR: (400 MHz, DMSO-d₆): δ 0.83 (t, 3H), 1.0 (d, 3H), 1.05 (broad, 24H), 1.20 (m, 2H), 2.25 (m, 2H), 3.4-3.6 (broad, C—H sugar), 3.8 (s, 3H), 4.15 (s, 1H), 4.58 (s, 1H), 4.75 (m, 2H), 5.0 (m, 1H), 5.18 (dd, 1H), 5.4 (d, 1H), 5.48 (d, 1H), 6.14 (m, 1H), 6.18 (s, 1H), 7.0 (m, 3H), 9.15 (s, 1H), 12.05 (s, 1H) ppm.

### Example 3

The acylation of esculin (0.75 g, 2 mmol) with palmitic acid (0.523 g, 2 mmol) was carried out as described in Example 1. Liquid chromatographic analysis showed that 48% of the acyl donor had been consumed after 48 hours. Using the same purification procedure as before, the esculin monopalmitate was obtained by liquid/liquid extraction. The structure of the esculin ester was confirmed by ¹H-NMR analysis:

¹H-NMR: (400 MHz, DMSO-d₆): δ 0.8 (t, 3H), 1.15 (broad, 24H), 1.4 (m, 2H), 2.25 (m, 2H), 3.2 (m, 1H), 3.65 (m, 1H), 4.1 (dd, 1H), 4.35 (d, 1H), 4.85 (d, 1H), 5.25 (s, 1H), 5.35 (d, 1H), 6.2 (d, 1H), 6.8 (s, 1H), 7.3 (s, 1H), 7.85 (d, 1H) ppm.

### Example 4

An acylation of rutin with lauric acid was carried out in a 27 ml reactor. Rutin (100 mg, 0.16 mmol) and lauric acid (20 mg, 0.10 mol) were dissolved in 20 ml dried tert.amylalcohol at 60° C. A controlled water content below 100 mM was adjusted and maintained in the reaction medium by addition of molecular sieves (4 g). The esterification reaction was started by addition of 0.2 g lipase of Candida antarctica (Novozym 435).

HPLC analysis showed that the conversion of rutin to the monoester amounted to 76%.

### Example 5

The acylation of esculin (100 mg, 0.27 mmol) with lauric acid (54 mg, 0.27 mmol) was carried out as described in Example 4.

HPLC analysis showed a conversion to the esculin monoester of 82%.

### Example 6

The acylation of esculin (100 mg, 0.27 mmol) with 11-aminoundecanoic acid (55 mg, 0.27 mmol) was carried out as described in Example 4.

HPLC analysis showed a conversion to the monoester of 61%.

### Example 7

The acylation of esculin (100 mg, 0.27 mmol) with 11-mercaptoundecanoic acid (59 mg, 0.27 mmol) was carried out as described in Example 4.

The conversion of esculin to the monoester amounted to 68% (HPLC analysis).

### Example 8

The acylation of esculin (100 mg, 0.27 mmol) with adipic acid (40 mg, 0.27 mmol) was carried out as described in Example 4.

HPLC analysis showed a conversion to the esculin monoester of 70%.

### Example 9

The acylation of rutin (100 mg, 0.16 mmol) with dodecane diacid (38 mg, 0.16 mmol—equimolar) was carried out as described in Example 4.

HPLC analysis showed a conversion to the rutin monoester of 75%.

### Example 10

The acylation of rutin (100 mg, 0.16 mmol) with dodecane diacid (754 mg, 3.27 mmol—acid excess) was carried out as described in Example 4.

HPLC analysis showed a 75% conversion of the rutin, the ratio of diesters to monoesters being 4:1.

### Example 11

The reaction of rutin (in an excess) with dodecane diacid was carried out in a 250 ml reactor.

Rutin (8, 13 mmol) and dodecane diacid (0.3 g, 1.3 mmol) were dissolved in 200 ml tert.amylalcohol and heated in vacuo (105-200 mbar) to 60° C. The vapours formed were passed trough a column filled with molecular sieves and recovered. In this way, a small water content of less than 100 mM was obtained in the reactor after a few hours. 2 g Candida antarctica lipase (Novozym 435) were then added.

A conversion of dodecane diacid to dodecane dioyl dirutin (2 molecules of rutin joined by a diacid molecule) and to dodecane dioyl rutin (monoester) of 100% was achieved after 4 days (HPLC analysis).

### Example 12

The acylation of esculin (8, 21.7 mmol) with dodecane diacid (5 g, 21.7 mmol—acid excess) was carried out as described in Example 11.

HPLC analysis showed that the conversion to the esculin monoester amounted to 92%.

### Example 13

The acylation of rutin (100 mg, 0.16 mmol) with hexadecane diacid (47 mg, 0.16 mmol) was carried out as described in Example 4.

87% of the rutin were converted (HPLC analysis).

The purification method of liquid/liquid extraction according to Example 1 enables hexadecane dioyl rutin (monoester) to be recovered. The structure of the product was confirmed by ¹H-NMR analysis:

¹H NMR: (400 MHz, DMSO d₆): δ 0.76 (d, 3H), 1.2 (m, 60H), 1.5 (m, 12H), 2.2 (m, 12H), 3.1-3.6 (broad, 8H), 3.7 (d, 1H), 4.45 (s, 1H), 4.65 (t, 1H), 5.43 (d, 1H), 6.18 (d, 1H), 6.36 (d, 1H), 6.84 (d, 1H), 7.50 (m, 2H) ppm.

### Example 14

The acylation of esculin (100 mg, 0.27 mmol) with hexadecane diacid (78 mg, 0.27 mmol) was carried out as described in Example 4.

HPLC analysis showed a conversion to the esculin monoester of 89%.

### Example 15

The reaction of esculin with thiooctanoic acid was carried out in a 250 ml reactor. Esculin (0.87 g, 2.5 mmol) and thiooctanoic acid (1.23 g, 6 mmol) were dissolved in 250 ml tert.amylalcohol and heated in vacuo (150-200 mbar) to 60° C. The vapors formed were passed through a column filled with molecular sieves and recovered. In this way, a small water content of less than 100 mM was obtained in the reactor after 21 hours. 2.5 g Candida antarctica lipase (Novozym 435) were then added.

After 70 hours, 50% of the esculin had been converted (HPLC analysis).

After the reaction, the enzyme was filtered and the reaction medium was concentrated by evaporation of the solvent. To remove excess thiooctanoic acid, a mixture of water, heptane and acetonitrile (2:3:4, v:v:v) was used for extraction, after which the ester was recovered by extraction with dichloromethane. The structure of the ester was verified by ¹H NMR:

¹H NMR: (400 MHz, DMSO d₆): 1.2-1.9 (broad, 8H), 2.1-2.4(broad, 4H), 3.2 (m, 2H), 3.5 (m, 1H), 3.7 (m, 1H), 4.12 (dd, 1H), 4.35 (d, 1H), 4.85 (d, 1H), 5.23 (d, 1H), 5.33 (d, 1H), 6.26(d, 1H), 6.84 (s, 1H), 7.33 (s, 1H), 7.86 (d, 1H) ppm.

### Example 16

The reaction of rutin with phenylpropionic acid was carried out in a 250 ml reactor. Rutin (8 g, 13 mmol) and penylpropionic acid (10 g, 67 mmol) were dissolved in 200 ml tert.amylalcohol and heated in vacuo (150-200 mbar) to 60° C. The vapors formed were passed through a column filled with molecular sieves and recovered. In this way, a small water content of less than 100 mM was obtained in the reactor after 17 hours. 13 g Candida antarctica lipase (Novozym 435) were then added.

HPLC analysis of the product showed a conversion to the rutin monoester of 55% after a reaction time of 105 hours.

1-18. (canceled)

19. A process for the enzymatic synthesis of esters of flavonoids and flavonoid derivatives, comprising the steps of
a) forming a reaction medium containing an organic solvent, a glycosylated flavonoid, aglycon flavonoid or flavonoid derivative, an acyl group donor, and an enzymatic catalyst under conditions in which the concentration of water is below 150 mM prior to the start of the reaction,
b) removing water and alcohol, if any, formed during the course of the reaction in order to maintain their concentration below 150 mM, and
c) separating the obtained flavonoid ester from the other reaction medium components.

20. A process according to claim 19, wherein the concentration of water in step a) is below 100 mM and the concentration of water and alcohol in step b) is maintained below 100 mM.

21. A process according to claim 19, further comprising adjusting the molar ratio of flavonoid to acyl donor in the reaction medium so that it is in the range from 0.01 to 20.00 during the reaction.

22. A process according to claim 19, further comprising adjusting the molar ratio of flavonoid to acyl donor in the reaction medium so that it is in the range from 0.02 to 10.00 during the reaction.

23. A process according to claim 19, further comprising continuously or periodically adding to the reaction medium during the course of the reaction further quantities of at least one constituent of the reaction medium.

24. A process according to claim 19, further comprising continuously or periodically removing at least one constituent of the reaction medium and, after fractionation, returning it to the reaction medium.

25. A process according to claim 24, wherein the entire reaction medium is periodically or continuously removed during the reaction and, after fractionation, one or more constituents of the medium removed are re-injected into the reactor.

26. A process according to claim 19, wherein, during the reaction, the temperature is adjusted to 20 to 100° C., the partial pressure over the reaction medium is adjusted to 10 mbar to 1,000 mbar and the reaction medium is stirred.

27. A process according to claim 19, wherein, residual flavonoids or residual acyl donor are separated from the obtained flavonoid esters by extraction with organic solvents or supercritical fluids, by distillation, by crystallization, by adsorption or by precipitation.

28. A process according to claim 19, further comprising fractionating the obtained flavonoid esters by precipitation or chromatography.

29. A process according to claim 19, wherein the flavonoid is selected from the group consisting of chalcone, flavone, flavanol, flavanone, anthocyan, flavanol, coumarin, isoflavone and xanthone.

30. A process according to claim 19, wherein the acyl donor is selected from the group consisting of a linear or branched, saturated, unsaturated or cyclic aliphatic acids containing up to 22 carbon atoms and optionally substituted by one or more substituents selected from the group consisting of hydroxyl, amino, mercapto, halogen and alkyl-S,S-alkyl, linear or branched, saturated or unsaturated aliphatic diacids containing up to 22 carbon atoms, a cinnamic acid optionally substituted by one or more substituents selected from the group consisting of hydroxyl, nitro, alkyl, alkoxy and halogen atoms, and a benzoic acid optionally substituted by one or more substituents selected from the group consisting of hydroxyl, nitro, alkyl, alkoxy and halogen atoms, or methyl, ethyl, propyl or butyl esters thereof.

31. A process according to claim 19, wherein the acyl donor is selected from the group consisting of palmitic acid, 1 6-hydroxyhexadecanoic acid, 12-hydroxystearic acid, 11-mercaptoundecanoic acid, thiooctanoic acid, china acid, 3,4-dihydroxycinnamic acid, 4-hydroxy-3-methoxycinnamic acid or 4-hydroxycinnamic acid.

32. A process according to claim 19, wherein the acyl donor is selected from the group consisting of hexadecane diacid or azelaic acid, an arylaliphatic acid and a dimeric acid derived therefrom.

33. A process according to claim 19, wherein the organic solvent is selected from the group consisting of propan-2-ol, butan-2-ol, isobutanol, acetone, propanone, butanone, pentan-2-one, ethane-1,2-diol, butane-2,3-diol, dioxan, acetonitrile, 2-methylbutan-2-ol, tert.butanol, 2-methylpropanol and 4-hydroxy-2-methylpentanone, aliphatic hydrocarbons, and mixtures of at least two of these components.

34. A process according to claim 19, wherein the organic solvent is the acyl donor.

35. A process according to claim 19, wherein the enzymatic catalyst comprises an immobilized protease, lipase or a combination thereof.

36. A process according to claim 19, wherein the enzymatic catalyst is selected from the group consisting of Candida antarctica, Rhizomucor miehei, Candida cylindracea, Rhizopus arrhizus or a combination thereof.

37. A process according to claim 19, wherein the water and the alcohol are removed from the medium by molecular sieves.

38. A process according to claim 19, wherein the water and the alcohol are removed from the reaction medium by pervaporation.
